(19) United States
(12) Patent Application Publication
(10) Pub. No.: US 2010/0030303 A1
(43) Pub. Date: Feb. 4, 2010

(54) MONITORING AMBIENT NOISE ON COMMUNICATION CHANNELS USED TO COMMUNICATE WITH MEDICAL DEVICES

(75) Inventors: Gregory J. Haubrich, Champlin, MN (US); Gary Kivi, Maple Grove, MN (US)

Correspondence Address: Medtronic, Inc., 710 Medtronic Parkway, Minneapolis, MN, 55432, US

(73) Assignee: Medtronic, Inc., Minneapolis, MN (US)

(21) Appl. No.: 12/182,691

(22) Filed: Jul. 30, 2008

(51) Int. Cl. A61N 1/00

(52) U.S. Cl. 607/60

(57) ABSTRACT

A medical device for wirelessly communicating with at least one implantable medical device (IMD) comprises a telemetry circuit that monitors an ambient noise on one or more communication channels used for the wireless communication with the at least one IMD. The medical device also includes a network interface that transmits data corresponding to the monitored ambient noise to a data storage device external from the device. The data storage device may retrieve and present the data corresponding to the monitored ambient noise received from one or more medical devices to facilitate identification or characterization of noise sources, designing or updating medical devices to compensate for ambient noise, or troubleshooting or otherwise analyzing operation of the medical devices. In some cases, the data storage device may automatically analyze the ambient noise to and, for example, provide alerts relating to ambient noise or medical device operation.

### TECHNICAL FIELD

The invention relates to medical devices and, particularly, communicating with medical devices.

### BACKGROUND

A variety of implantable medical devices for delivering a therapy and/or monitoring a physiological condition have been clinically implanted or proposed for clinical implantation in patients. Implantable medical devices, or IMDs, may deliver electrical stimulation or fluid therapy to, and/or monitor conditions associated with, the heart, muscle, nerve, brain, stomach or other organs or tissue. This therapy and/or monitoring may occur according to a program contained within the IMD. A program may comprise respective values for each of a plurality of parameters, specified by a clinician. The clinician may specify the parameters of the program through use of a clinician programming device or programmer, often located at an office of the clinician, to communicate the program wirelessly to the IMD.

The clinician may also retrieve information from the IMD using the programming device. The information may be collected by the IMD over time. As examples, the information may relate to the condition of the patient, such as trends of physiological parameters over time, or the condition of the IMD or related components, such as battery voltage or impedance, or lead impedance.

A patient may also receive a patient programmer or programming device, or home monitor, that may also communicate wirelessly with the IMD implanted in that patient. The patient programmer or home monitor may communicate with the patient's IMD to, as examples, retrieve information concerning the application of a therapy, update one or more programs contained within the patient's IMD, or perform other actions pertinent to monitoring the patient or the patient's IMD. In some instances, the patient programmer or home monitor is connected to a network, and through this network connection, the clinician and other authorized users may access the patient programmer or home monitor to remotely retrieve information (e.g., collected over time) concerning the application of the therapy, patient physiological parameters or IMD parameters, update the one or more programs, or perform other operations to monitor, program or otherwise interact with the patient's IMD.

### SUMMARY

In general, this disclosure is related to monitoring wireless communication environments. In particular, a device for wirelessly communicating with a medical device may monitor communication channels used to wirelessly communicate with the medical device. The medical device may be implanted in a patient and referred to as an implantable medical device (IMD). Typically, the device for wireless communication comprises a home programmer or monitor device, although it may be a clinician programming device. This programming device may monitor the wireless communication environment to collect data concerning ambient noise on each channel, e.g., range of frequencies, used or available for the wireless communication. The programming device may then, after monitoring or collecting this data, transmit the data to a data storage device remote from the programming device.

The data storage device may store data concerning the ambient noise of a plurality of devices for wirelessly communicating with IMDs, referred to herein as programming devices. The data storage device may organize or otherwise index this data to enable manual or user inspection of the data. The data storage device may also automatically analyze the collected data and generate warning, alerts, or errors based on the analysis to the programming device or any other computing device accessible via the network, such as a computing device associated with a clinician who treats the patient in whom the IMD that the programming device communicated with is implanted. The data storage device may also automatically, through the above analysis, detect failures or malfunctions in wireless communications conducted by the programming device, such as a malfunctioning antenna. A user may interact with data storage device to cause a home programmer device to initiate a sight survey, e.g., the collection of data concerning ambient noise, so as to troubleshoot or otherwise view current ambient noise conditions. These and other actions may, for example, improve wireless communications between programming devices and IMDs.

In one embodiment, a method comprises monitoring, with a device that wirelessly communicates with at least one implantable medical device (IMD), an ambient noise on one or more communication channels used for the wireless communication with the at least one IMD, and transmitting data corresponding to the monitored ambient noise via a network to a data storage device remote from the device that wirelessly communicates with the at least one IMD.

In another embodiment, a medical device for wirelessly communicating with at least one implantable medical device (IMD). The medical device comprises a telemetry circuit that monitors an ambient noise on one or more communication channels used for the wireless communication with the at least one IMD, and a network interface that transmits data corresponding to the monitored ambient noise via a network to a data storage device remote from the device that wirelessly communicates with the at least one IMD.

In another embodiment, a system comprises at least one implantable medical device (IMD), a network, a data storage device and a medical device for wirelessly communicating with the at least one IMD. The medical device comprises a telemetry circuit that monitors an ambient noise on one or more communication channels used for the wireless communication with the at least one IMD and a network interface that transmits data corresponding to the monitored ambient noise via the network to the data storage device external from the device that wirelessly communicates with the at least one IMD.

In another embodiment, a computer-readable medium comprising instructions that cause a programmable processor to monitor, with a device that wirelessly communicates with at least one implantable medical device (IMD), an ambient noise on one or more communication channels used for the wireless communication with the at least one IMD, and transmit data corresponding to the monitored ambient noise via a network to a data storage device remote from the device that wirelessly communicates with the at least one IMD.

The details of one or more embodiments of the invention are set forth in the accompanying drawings and the description below. Other features, objects, and advantages of the invention will be apparent from the description and drawings, and from the claims.

### BRIEF DESCRIPTION OF DRAWINGS

FIG. 1 is a block diagram illustrating an example system 10 in which one or more of programming devices monitor ambient noise.

FIG. 2 is a schematic diagram illustrating a portion of the system of FIG. 1 in more detail.

FIG. 3 is a functional block diagram illustrating various components of an IMD in an example configuration.

FIG. 4 is a functional block diagram illustrating various components of a programming device according to one example configuration.

FIG. 5 is a flowchart illustrating an example interaction between a programming device and a data storage device.

FIG. 6 is a flowchart illustrating an example operation of a programming device in monitoring ambient noise.

### DETAILED DESCRIPTION

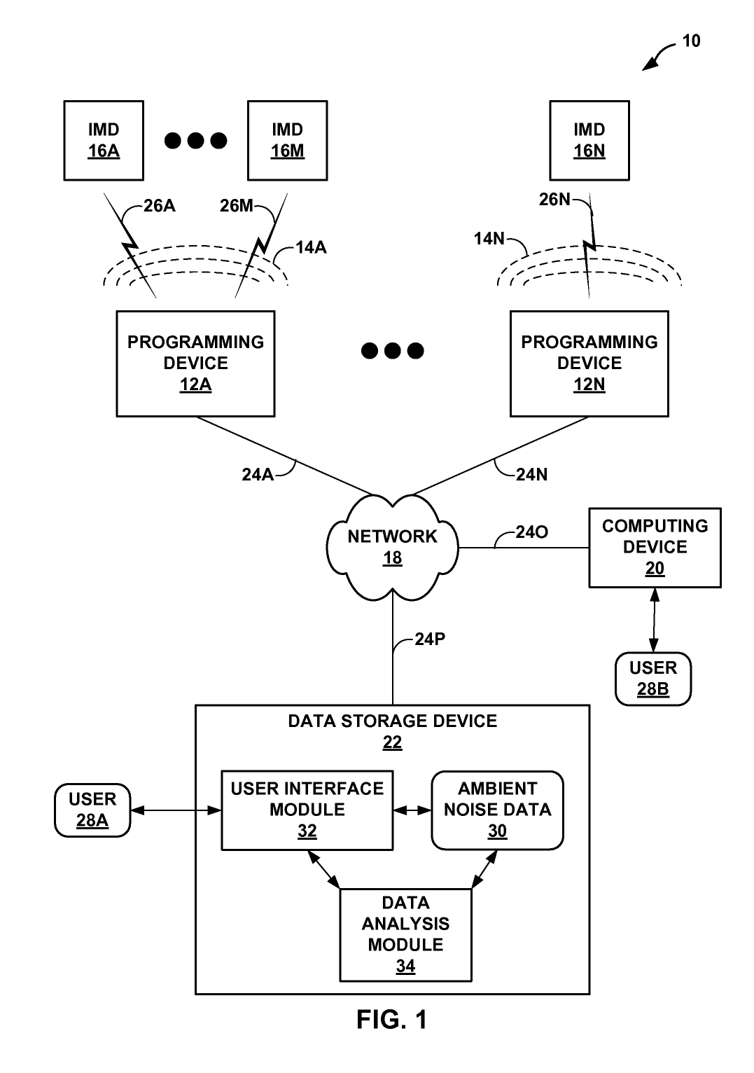

FIG. 1 is a block diagram illustrating an example system 10 in which one or more of programming devices 12A-12N (“programming devices 12”) monitor ambient noise 14A-14N (“ambient noise 14”). As shown in FIG. 1, system 10 includes a plurality of implantable medical devices (IMDs) 16A-16N (“IMDs 16”), a network 18, at least one computing device 20 and a data storage device 22. Programming devices 12 and IMDs 16 may be substantially co-located, and also may be located remotely from computing device 20 and data storage device 22. Computing device 20 and data storage device 22 may be substantially co-located or located remotely from each other. By way of communication links 24A-24P (“communication links 24”), each of programming devices 12, computing device 20 and data storage device 22 may interconnect and communicate with one another via network 18.

Programming devices 12 may each communicate with respective IMDs 16. In the example of FIG. 1, programming device 12A communicates with IMDs 16A-16M via wireless communication channels 26A-26M, although not necessarily at the same time. Programming device 12N communicates with IMD 16N via wireless communication channel 26N. In this respect, each of programming devices 12 may represent a device that wirelessly communicates, or is at least capable of wireless communication, with at least one implantable medical device (IMD). Although illustrated in FIG. 1 as communicating with IMDs 16 via a respective channel 26, in some examples a programming device 12 may communicate with any given IMD 16 via a selected one of a plurality of available channels 26, and may communicate with multiple IMDs 16 via the same channel 26 at different times.

Each of IMDs 16 may represent any device that may be implanted in a patient to monitor and/or deliver therapy to the patient in whom each of IMDs 16 is implanted. As examples, IMDs 16 include pacemakers, carioverters, defibrillators, pacemaker-cardioverter-defibrillators, neurostimulation devices, or any other device that is implanted to treat a condition, including cardiac arrhythmia, tremor, Parkinson's disease, epilepsy, urinary or fecal incontinence, sexual dysfunction, obesity, or gastroparesis. While described in this disclosure with respect to IMDs 16, the techniques may apply to any medical device, implantable or otherwise, capable of wireless communication.

Each of programming devices 12 may comprise a general purpose computing device (e.g., a desktop computer, a laptop computer, a workstation, a personal digital assistant, or a cellular phone) that executes instruction, software, or a computer program to cause each of devices 12 to wirelessly communicate with respective IMDs 16 via wireless communications channels 26. Alternatively, programming devices 12 may comprise a computing device dedicated to establishing wireless communication with at least one IMD, such as one or more of IMDs 16, and monitoring and/or programming the at least one IMD via the wireless communication.

A programming device 12 may initiate such communications to update, edit or otherwise tailor the configuration one or more of the IMDs 16. In this regard, programming devices 12 may “program” each of respective IMDs 16. While described herein with respect to programming devices, these devices may represent other devices that only monitor and/or both monitor and program respective IMDs 16. Those devices that only monitor IMDs 16 may be referred to herein as “monitor” devices, and sometimes referred to as home monitors. Thus, the techniques described in this disclosure should not be limited to communication devices 12 that program an IMD 16.

Programming devices 12 may be referred to as “remote” in that each of these programming devices 12 may be located distant from data storage device 22 and computing device 20, such as within a clinician's office. Often, programming devices 12 may be located at a home of a patient in which one or more IMDs, e.g., one or more of IMDs 16, have been implanted. In this case, programming devices 12 may be referred to as a “home programming device” or “home monitor device” to reflect the location of the programming device. Programming devices 12 may be also each referred to, for short, as “remote programmers” or “home programmers” (or “remote monitors” or “home monitors”).

Computing device 20 may also comprise a general purpose computing device (e.g., a desktop computer, a laptop computer, a workstation, a personal digital assistant, or a cellular phone) or any other device capable of interfacing with programming devices 12 via network 18. That is, computing device 20 may comprise a general purpose computing device that includes a control unit or programmable processor. This general purpose computing device may also include instructions, software, or a computer program that causes the programmable processor or control unit to interface with programming devices 12 via network 18. Computing device 20 may, alternatively, comprise a device dedicated to interfacing with programming devices 12 and/or IMDs 16.

Computing device 20 may, in some cases, be located at an office of a clinician. Computing device 20 may interface with remote programming devices 12, such as programming devices located at the home or other location of the patient, via network 18 to remotely cause programming devices 12 to monitor and/or program IMDs 16. In some cases, computing device 20 may be a clinician programming device or, for short, a “clinician programmer.” In other cases, one or more of remote programming devices 12 may comprise a clinician programming device, while a different one or more of remote programming devices 12 comprise home programmer or monitor devices. Computing device 20 may be used by a clinician, or technician of a manufacturer of IMDs 16 and/or programming devices 12 to interact with such remotely located devices. Computing device 20 may communicate over network 18 with one or more of programming devices 12 using one or more of a plurality of protocols depending on those of the plurality of protocols supported by network 18.

Commonly, network 18 comprises a packet-based computer network that supports an Internet Protocol (IP)/Transmission Control Protocol (TCP). In a packet-based computer network, computing device 20 may establish a communication session over network 18 with a destination, e.g., remote programmer device 12A, via links 24O and 24A. Computing device 20 may then transmit data over this session by dividing the data into discreet data units, referred to as “packets.” The packets traverse link 24O and arrive at network 18. Network 18, which may comprise a plurality of computing devices that route, switch or otherwise direct this traffic, may forward the packets across network 18 to link 24A. Remote programming device 12A may, upon receiving the packets, reassemble the data and respond to this data with its own in a reverse process.

Network 18, as described above, may comprise one or more network devices, such as a router, a switch, and a hub, to direct, route or otherwise forward traffic, e.g., the distinct data units, across network 18. Network 18 may comprise a public network, such as the Internet, or a private network, such as those maintained or operated by an enterprise or business. While described in this disclosure with respect to a general network, such as network 18, system 10 may be implement a network similar in form and functionality to that provided by the Medtronic CareLink® Network developed by Medtronic, Inc., of Minneapolis, Minn. In this type of network, programming devices 12 may each comprise a home monitor or home monitor device for monitoring one or more IMDs, similar to IMDs 16. Computing device 20 may, again in this type of network, represent a clinician programming device that is capable of programming and/or retrieving data from the home monitors and/or IMDs 16.

Although shown as a single network 18 in FIG. 1, network 18 may comprise a plurality of networks, each interconnected to form one large network. In this instance, one or more of the plurality of networks may comprise a private network and one or more of the plurality of networks may comprise a public network. Some of the private networks may be interconnected across the public networks by services such as a virtual private network service (VPN) and a virtual private large area network (LAN) service (VPLS). Often such communications between the private networks are encrypted or otherwise secured to maintain privacy of the data communicated across the public network.

Considering the often confidential nature of patient data monitored by IMDs 16 and relayed by programming devices 12 via network 18, network 18 may support such confidential communications. Programming devices 12 and computing device 20 may therefore communicate over network 18 using security or encryption protocols, such as a secure socket layer (SSL) protocol, a transport layer security (TLS) protocol, or any other protocol that encrypts or otherwise secures data. Moreover, programming devices 12 and computing device 20 may reside within the same virtual private network or other private network operating over a public network so as to preserve the confidentially of such communications from leaking onto the public network.

Communication links 24A-24P (“links 24”) may comprise one or more communication lines, such as coaxial cable, fiber optical cable, copper cable, or any other physical medium by which communications are normally transmitted. Alternatively, links 24, although not illustrated in FIG. 1, may represent wireless communication links by which wireless communication may occur with network 18 according to, for example, any of the 802.11x family of wireless communication protocols. Computing device 20 may therefore communicate with programming devices 12 over network 18 via wireless or wired communication links 24 in order to remotely monitor and/or program IMDs 16.

While not shown in FIG. 1, computing device 20 may, similar to programming devices 12, wirelessly communicate with an IMD 16 located proximate to computing device 20 via a wireless communication channel 26. Thus, computing device 20 may also interface directly with one or more IMDs 16 to monitor or program one or more IMDs.

However, establishing a wireless communication with an IMD directly often requires that the IMD be located proximate to computing device 20. That is, wireless communication may be limited in range due in part to attenuation of the wireless communication signal, and interference on wireless communication channels used to communicate wirelessly. Thus, a patient, in whom one of IMDs 16 may be implanted, typically must travel to the clinician's office in order to enable the direct wireless communication. To reduce travel, especially for patients that require frequent monitoring and/or programming of the IMD, computing device 20 may remotely interact with a remote programming device 12, e.g., home monitor, to monitor and/or program the IMD 16, as well as, interact with remote programming devices 12 to update, monitor and/or program these devices 12.

Interference may occur with respect to wireless communications initiated by programming devices 12. “Interference,” as used in this disclosure, may refer to ambient noise, such as ambient noise 14A-14N (“ambient noise 14”). Typically, any number of sources may generate ambient noise 14, including devices, such as cellular phones, cellular towers, cordless telephones, wireless access points or routers, radio communication devices, two-way radios, walkie-talkies, police-band radios, walkmans, Apple Inc.'s iPod, Bluetooth™ enabled devices and peripherals, computers, monitors, high-definition television (HDTV) towers, televisions, stereos, and weather balloons. This interference or ambient noise 14 may occur across a broad swath of frequencies, some of which may interfere with a range of frequencies used by remote programming devices 12 to wireless communicate with IMDs 16.

The range of frequencies commonly used by programming devices 12 to communicate with IMDs 16 may comprise a frequency range of 402 megahertz (MHz) to 405 MHz. This frequency range may be referred to as a radio frequency (RF) range as this range lies in a range of frequencies assigned to radios. This frequency range may also be referred to as a medical implant communication service (MICS), which is the name of one specification for using this frequency range to communicate with IMDs 16. Likewise, ambient noise 14 that interferes with this RF range may be referred to herein as RF ambient noise or MICS ambient noise. While described herein with respect to the MICS frequency range, the techniques may apply to any other frequency ranges used for communicating with medical devices, including frequencies defined by Medical Data Service (MEDS), e.g., so-called “lower” MEDS frequencies 401-402 MHz and “upper” MEDS frequencies 405-406 MHz, any other frequency used for wireless telemetric communication.

This range of frequencies may be further divided into discreet channels. A channel may comprise a sub-range of the range of frequencies. Assuming the above range of frequencies for purposes of illustration, the range of 402-405 Mhz may be divided into ten (10) channels, each channel comprising a sub-range spanning a total of 300 kilohertz (kHz). For example, one channel may comprise a sub-range of 402.0 Mhz through 402.3 Mhz. Each channel may further include a guard band, which buffers both sides of the channel to decrease, lessen, or otherwise dampen intra-channel interference or interference between adjacent channels. Again, assuming a set sub-range of 300 kHz, a first 40 kHz guard band may buffer the lowest 40 kHz portion of each channel and a second 40 kHz guard band may buffer the highest 40 kHz portion of each channel. Each channel, in this instance, may therefore comprise a 220 kHz band over which wireless communications may occur. Wireless communications channels 26 may, for example, each represent one of these 220 kHz channels or any other band over which wireless communication may occur.

Prior to establishing a wireless communication with an IMD 16, a programming device 12 (and, in some examples, computing device 20) may scan each channel of the range of frequencies to determine on which channel to establish a wireless communication session, or in other words, to coordinate the wireless communication, with one of IMDs 16. Programming devices 12 may scan each of the channels to determine on which channel to establish the wireless communication session by determining, for each of the channels, a level of ambient noise or interference. Programming device 12A, for example, may monitor each of the above 10 channels prior to establishing a wireless communication with IMD 16A. Programming device 12A may monitor, for each of the channels, a level of ambient noise 14A within the sub-range of the range of frequencies corresponding to that channel.

Upon determining a level of ambient noise 14 for each of the channels, programming devices 12 may determine which of the channels has the lowest level of interference (or in other words, a least amount of ambient noise 14 interfering with that sub-range of the range of frequencies defined by that channel) and select this lowest relative noise channel for establishing the wireless communication. Once the lowest relative noise channel is selected, programming devices 12 may coordinate with IMDs 16 to wirelessly communicate via the selected channel. Each of wireless communication channels 26 may represent this selected lowest relative noise channel.

In some instances, programming devices 12 may be configured to compensate for particularly noisy environments. For example, programming devices 12 may execute algorithms designed to compensate for ambient noise 14 generated by devices using the same or overlapping range of frequencies as that of programming devices 12. The algorithm may identify patterns of such ambient noise 14 and filter these patterns to improve wireless communications between IMDs 16 and programming devices 12. As new sources of ambient noise 14 are frequently emerging, these algorithms or other measures designed to compensate for ambient noise 14 may become outdated and ineffective in that the new sources may generate patterns not identifiable and, as a result, compensated for by the algorithm or other measures. This interference may degrade or impact, if not prevent, wireless communications between programming devices 12 and IMDs 16.

In some instances, one or more of programming devices 12 may monitor or collect data corresponding to respective ambient noise 14 on one or more communication channels, e.g., such as one or more of the above described 10 MICS channels, used for the wireless communication with the at least one IMD, e.g., one or more of respective IMDs 16. One or more of programming devices 12 may also monitor a quality of wireless communication with one or more of respective IMDs 16. This monitoring may be referred to as monitoring a “quality of service” of the wireless communication session or connection.

To monitor the quality of service, one or more of programming devices 12 may monitor a number of wireless communication connections or sessions dropped, a number of wireless communication connections attempted and a number of wireless connections successfully completed with one or more of respective IMDs 16. One or more of programming devices 12 may also monitor the quality of service by monitoring throughput or an amount of data received from one or more IMDs 16 compared to an amount of time to receive the data from the one or more IMDs 16.

One or more of programming devices 12 may also measure the quality of service by monitoring a signal-to-noise ratio during an established wireless communication connection or session. To measure the signal-to-noise ration, the programming device measures the signal when the IMD is transmitting and the noise is measured by the programming device during a turn-around time when neither the IMD nor the programming device are transmitting. One or more of programming devices 12 may also measure the below described signal strength indicator, which represents a quantized average signal-to-noise ration.

This ambient noise and quality of service monitoring may occur at set times or according to a schedule and, in these instances, may be referred to as “periodic monitoring.” Alternatively or in conjunction with the periodic monitoring, the monitoring may occur prior to communications, as described above, and may be referred to as “pre-communication monitoring.” In some instances, the monitoring may occur during a communication session, and this form of monitoring may be referred to as “session monitoring.” While monitoring in any one of the above ways, one or more of programmers 12 may timestamp or otherwise mark or indicate the date and time at which the data was monitored or collected.

Regardless of when the monitoring may occur, each of programming devices 12 may transmit the data corresponding to monitored ambient noise 14 and/or the quality of service to a data storage device located remotely from the one or more of programming devices 12, e.g., data storage device 22. Alternatively, data storage device 22 may retrieve the data. As above, this transmission of data or retrieval of data may occur according to a schedule or at set times. For example, data storage device 22 may retrieve the data every 15 days. In some cases, programming devices 12 or data storage device 22 may transmit the data to a computing device (e.g., a clinician programmer or computing device 20) of a clinician responsible for the care of the patient in whom a respective one of IMDs 16 is implanted. While described below with respect to data storage device 22, any device remote from one of programming devices 12 may receive and store the data.

In the example of FIG. 1, data storage device 22 may be coupled to each of programming devices 12 via network 18 and links 24A-24N and 24P. Programming devices 12 may therefore transmit the data via network 18 to data storage device 22. While shown as coupling via network 18 to remote programmer devices 12, data storage device 22 may be coupled directly to one or more programming devices 12 or computing device 20.

Data storage device 22 may comprise any device capable of storing or collecting data, such as a desktop computer, laptop computer, a workstation, a server, or any combination thereof. Typically, data storage device 22 represents a server that stores large amounts of data in accordance with a structured format, such as a database defined by a database schema. Data storage device 22 may, in this instance, comprise a network server that stores ambient noise data 30 to a database locally hosted by the network sever. Data storage device 22 may analyze the data stored to the database, in this case, to construct indexes or otherwise arrange references to the data to enable a user, such as one of users 28A, 28B (“users 28”), to quickly access relevant data. As shown in FIG. 1, data storage device 22 includes ambient noise data 30, user interface module 32 and a data analysis module 34.

Upon receiving the data corresponding to ambient noise 14 and/or the quality of service from remote programming devices 12 via network 18, data storage device 22 may store this data as ambient noise data 30. Ambient noise data 30 may therefore store both data directly concerning ambient noise 14 and data that indirectly concerns ambient noise 14, such as the above described quality of service data. The data corresponding to the quality of service may indirectly relate to ambient noise 14 because this data reflects the affects of ambient noise 14 on the quality of wireless communications. In this respect, ambient noise data 14 may comprise both data corresponding to ambient noise 14 and the quality of service and “ambient noise data” may be used herein when discussing both types of direct and indirect data.

Ambient noise data 30 may be stored to a database or other data structure, such as a table or linked list, for storing data collected or monitored by programming devices 12 concerning ambient noise 14. Ambient noise data 30 may be organized or indexed according to geographical location (e.g., a location in which each of programming devices 12 resides), an identifier assigned to device 12 (e.g., an IP or media access control (MAC) address assigned to each of devices 12), a patient, an IMD (e.g., such as by which ones of IMDs 16 or types of IMDs in general with which each of programming devices 12 may interact), or any other classifiable characteristic of one or more IMDs 16, programming devices 12, and ambient noise data. Ambient noise data 30 may comprise historical data related to ambient noise 14 in that ambient noise data 30 may represent ambient noise data collected over a period of time.

User interface module 32 may represent any module capable of presenting a user interface with which a user, such as user 28A, may interact to at least view ambient noise data 30. User interface module 32 may enable a user, e.g., user 28A, to locally interface with data storage device 22 to view ambient noise data 30. User interface module 32 may also enable a user located remote from data storage device 22, e.g., user 28B, to interface with data storage device 22 to view ambient noise data 30. User interface module 32 may enable such remote viewing by serving a web page or otherwise enabling remote network-based access to data storage device 22. User 28B may cause computing device 20 to download or access this remote web page or other user interface to interact with data storage device 22 to view ambient noise data 30.

Via these interactions, users 28 may either locally or remotely analyze ambient noise data 30 by interacting via the same or a different user interface presented by user interface module 32 with data analysis module 34. As one example, users 28 may interface with data analysis module 34 to reorder or otherwise sort ambient noise data 30 by one or more of the above described classifiable characteristics. As another example, users 28 may interface with data analysis module 34 to restrict the data to a particular device 12 or channel of a particular device. Users 28 may also view ambient noise data 30 by a time during which the sample was taken, e.g., day or night. Users 28 may also view ambient noise data 30 by geographical location, e.g., based on known locations of programming devices 12 or IP addresses assigned to programming devices 12. Users 28 may view ambient noise data 30 to troubleshoot a particular one of remote programmer devices 12 and/or IMDs 16.

For example, users 28 may, by viewing ambient noise data 30, determine that one of programming devices 12 are malfunctioning in that the ambient noise data 30 for that particular one of devices 12 suggests no ambient noise 14 was detected. Users 28 may conclude from this silence that one or more of an antenna or transmitter of that particular one of devices 12 has malfunctioned.

User interface module 32 may further enable users 28 to configure data analysis module 34 to automatically analyze ambient noise data 30 and provide warnings or errors to users 28. These warnings or errors may be sent by email, text message or other communication medium to associated accounts of users 28 via network 18 and/or displayed as a message to users 28 via one of the user interfaces presented by user interface module 32. The warning or other errors may enable users 28 to quickly troubleshoot a particular one of programming devices 12 and/or IMDs 16. Data analysis module 34 may also be configured in other ways to automatically detect certain malfunctions and alert users 28 of the malfunction.

Ambient noise data 30, as described above, may also store information pertaining to the quality of service, such as a number of connections dropped by a particular one of remote programming devices 12. Users 28 may view this data to inspect ambient noise levels and patterns corresponding to those for which a high number of connections were dropped. Users 28 may then, using this information, update algorithms to account for new patterns caused by recently released ambient noise sources.

Users 28 may also determine, from the historical quality of service data stored as ambient noise data 30, that a particular one of programmers 12 may not contain all of the features necessary to compensate for ambient noise 14. This one of programmers 12 may be referred to as a “mid-range” or “low-end” programmer due to the lack of features. User 28 may then determine that this one of programmers 12 need be replaced with a programmer that provides these additional features to compensate for ambient noise 14. This programmer may be referred to as a “top-of-the-line” or “high-end” programmer. In this regard, the techniques described in this disclosure may enable users 28 to analytically determine which patients need a top-of-the-line programmer and, likewise, which no longer require a top-of-the-line programmer.

Again, data analysis module 34 may further be configured to automatically detect instances where a high number of connections are dropped and alert users 28 of the issue. For example, data analysis module 34 may analyze a number of connections dropped as compared to a number of connections attempted by remote programmer device 12A and determine that programming device 12A has dropped a high number of connections. Data analysis module 34 may, to make this determination, compare the above connections-dropped-to-connections-attempted metric to a configurable threshold. Data analysis module 34, in response to this determination, may issue an alert or error to users 28 via a user interface presented by user interface module 32. Data analysis module 34 may also issue an alert to programming device 12A, or more generally, any one of remote programming devices 12 for which the error or alert is relevant.

Data analysis module 34 may also analyze ambient noise data 30 to automatically determine whether interference is impacting communications, e.g., measure the quality of service. Data analysis module 34 may, for example, first determine in the manner described above those of programming devices 12 dropping a relatively high number of connections. Data analysis module 34 may then analyze ambient noise data 30 to characterize ambient noise 14 in which those identified programming devices 12 reside. Data analysis module 34 may then issue alerts to programming devices 12 suggesting, for example, that these programming devices 12 be moved away from the source of ambient noise 14. Data analysis module 34 may also identify the source of ambient noise 14 in the alert through characterization of the data corresponding to ambient noise 14.

Ambient noise data 30 may further provide a repository of data that enables users 28 to more adequately design future programmers or other devices that make use of wireless communication. For example, ambient noise data 30 may indicate that the above described “mid-range” devices or programmers do not adequately compensate for ambient noise 14 or conversely that these “mid-range” devices are overdesigned. Users 28 may then adjust the design of the mid-range programmers to account for the deficiencies or overdesign. In the case of overdesign, adjusting these programmers to reduce overdesign may lower a cost associated with these mid-range programmers.

As a result, ambient noise data 30 may enable users 28 to adjust algorithms to account for emerging patterns that may impact wireless communications between programming devices 12 and respective IMDs 16, thereby increasing the fidelity of wireless communications between such devices 12 and IMDs 16. Ambient noise data 30 may also enable automatic characterization and troubleshooting of wireless communications and the issuance of alerts or other warnings to suggest actions that mitigate the impact of ambient noise. Again, this may improve the fidelity of wireless communications between remote programming devices 12/computing device 20 and IMDs 16.

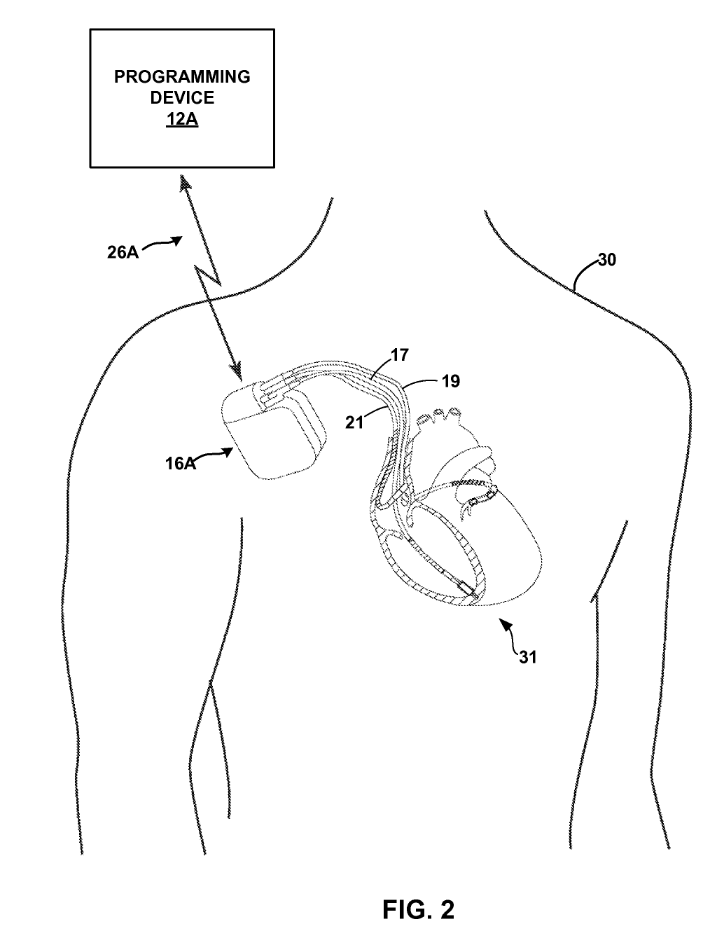

FIG. 2 is a schematic diagram illustrating a portion of system 10 of FIG. 1 in more detail. In particular, IMD 16A is shown in FIG. 2 implanted within a patient 30. In the illustrated example, IMD 16A is coupled to leads 17, 19 and 21 that extend from IMD 16A and into heart 31 of patient to position electrodes on the leads within the heart. IMD 16A monitors and/or applies stimulation therapy to patient 30 via the electrodes. In some examples IMD 16A may be a cardiac pacemaker, cardioverter, defibrillator, or pacemaker-cardioverter defibrillator. While described below with respect to IMD 16A and programming device 12A for ease of illustration purposes, each of IMDs 16 and programming devices 12 may be similarly situated, and comprise similar components to those described below with respect to IMD 16A and programming devices 12A.

IMDs 16 may comprise implantable electrical stimulators. IMDs 16 may provide electrical stimulation to treat any condition that may benefit from stimulation therapy. For example, IMDs 16 may be used to treat pain, tremor, Parkinson's disease, epilepsy, urinary or fecal incontinence, sexual dysfunction, obesity, or gastroparesis. In this manner, IMDs 16 may provide neurostimulation, spinal cord stimulation (SCS), deep brain stimulation (DBS), pelvic floor stimulation, gastric stimulation, or any other stimulation therapy. IMDs 16 may be coupled to one or more leads that position electrodes proximate to target tissue in a patient for receipt of stimulation for any such therapy.

Although FIG. 2 shows IMD 16A as implanted, other examples may include external medical devices that monitor and/or deliver therapy to a patient. While described in this disclosure with respect to an electrical stimulation device, IMDs 16 may comprise any type of IMD, including an IMD for monitoring a condition of a patient without delivering a therapy or an IMD for delivery of fluid therapies to patient 30. In addition, patient 30 is ordinarily, although not necessarily, a human patient.

In exemplary embodiments, IMD 16A may deliver stimulation therapy according to one or more programs. A program includes one or more parameters that define an aspect of the therapy delivered by IMD 16A according to that program. For example, a program that controls delivery of stimulation by IMD 16A in the form of pulses may define a voltage or current pulse amplitude, a pulse width, a pulse rate, for stimulation pulses delivered by IMD 16A according to that program. A program may also define other parameters of stimulation, such as duty cycles or times of day for stimulation, escape intervals for demand pacing, rate slopes for rate responsive pacing, or progressions of pacing and/or shocks for treating tachyarrhythmias, such as ventricular fibrillation.

A user, such as a clinician or patient 30 or users 28, may interact with a user interface of programming device 12A to program IMD 16A. In some examples, a user may interact with a user interface of computing device 20 (FIG. 1) and through communication between the computing device and programming device 12A, program IMD 16A. Programming of IMD 16A may refer generally to the generation and transfer of commands, programs, or other information to control the operation of IMD 16A. For example, programming device 12A may transmit programs, parameter adjustments, program selections, or other information to control the operation of IMD 16A, e.g., by wireless telemetry or wireless communication.

As described above, a user may also retrieve information from IMD 16A using programming device 12A (or through programming device 12A using computing device 20). The information retrieved from IMD 16A may include current operational parameters, as well as information collected by IMD 16A and stored therein over time. As examples, the stored information may include information regarding physiological parameters of patient 30 collected by the IMD over time, or system operation parameters, such as IMD battery or lead impedance, collected over time.

IMD 16A may be constructed with a biocompatible housing, such as titanium or stainless steel, or a polymeric material such as silicone or polyurethane. IMD 16A may be surgically implanted at a site in patient 30 selected based on, for example, surgical or lead routing convenience, or cosmesis, such as the pectoral location illustrated in FIG. 2. Alternatively, IMD 16A may be external with percutaneously implanted leads.

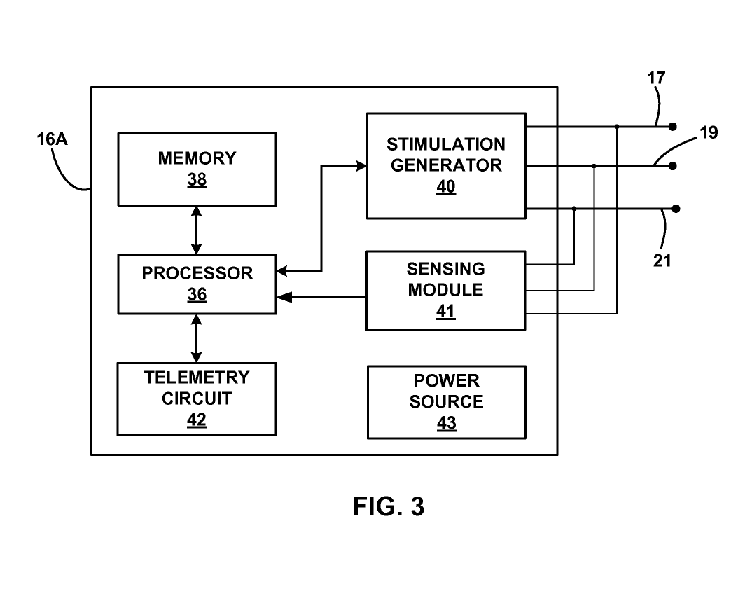

FIG. 3 is a functional block diagram illustrating various components of an IMD 16A in an example configuration. In the example of FIG. 3, IMD 16A includes a processor 36, a memory 38, a stimulation generator 40, a sensing module 41, a telemetry circuit 42, and a power source 43. Any IMD 16 may be similarly configured.

Memory 38 may store instructions for execution by processor 32, stimulation therapy data, programs, patient physiological parameter information, and any other information regarding therapy or patient 30. Information within memory 32 may be recorded for long-term storage and retrieval by a user, and the information may include any data created by or stored in IMD 16A. Memory 34 may include separate memories for storing instructions, information regarding patient physiological parameters, information regarding therapies delivered to a patient, and any other data that may benefit from separate physical memory modules. When executed by processor 36, instructions stored in memory 38 cause processor 36 and IMD 16A to perform any of the functions ascribed to them herein.

Processor 36 controls stimulation generator 40 to deliver electrical stimulation via, for example, combinations of electrodes on one or more leads 17, 19 and 21, e.g., as stimulation pulses or continuous waveforms. Stimulation generator 40 may include stimulation generation circuitry to generate stimulation pulses or waveforms and switching circuitry to switch the stimulation across different electrode combinations, e.g., in response to control by processor 36.

Sensing module 41 is also connected to electrodes on leads 17, 19 and 21, and may sense electrical signals within patient 30 (FIG. 2) via selected combinations of the electrodes, e.g., as controlled by processor 36. Sensing module 41 may comprises circuitry that conditions the electrical signals for processing by processor 36, such as one or more amplifiers or filters, or an analog-to-digital converter. In some examples, sensing module 41 may comprises circuitry that provides an indication to processor 36 of the occurrence of events in the signal, such as cardiac depolarizations in an electrogram. Processor 36 may store digitized versions of the sensed signals, information regarding event indications from sensing module 41, or other information derived from processing the sensed signal within memory 38 over time. Processor 36 may provide such information to a user, e.g., in response to a request, via telemetry circuitry 42 and a programming device 12.

Power source 43 delivers operating power to the components of IMD 16A. Power source 43 may include a small rechargeable or non-rechargeable battery and a power generation circuit to produce the operating power. Recharging may be accomplished through proximal inductive interaction between an external charger and an inductive charging coil within IMD 16A. In some embodiments, power requirements may be small enough to allow IMD 16A to utilize patient motion and implement a kinetic energy-scavenging device to trickle charge a rechargeable battery. In other embodiments, traditional batteries may be used for a limited period of time. As a further alternative, an external inductive power supply could transcutaneously power IMD 16A when needed or desired.

Wireless telemetry in IMD 16A with remote programmer device 12A may be accomplished by the above described radio frequency (RF) wireless communication with remote programmer device 12A. Telemetry circuit 42 may send information to and receive information from remote programmer device 12A on a continuous basis, at periodic intervals, or upon request from remote programmer device 12A. To support RF communication, telemetry circuit 42 may include appropriate electronic components, such as amplifiers, filters, mixers, encoders, decoders, and the like. Telemetry circuit 42 may include an antenna for receiving and transmitting the data.

Using telemetry circuit 42, IMD 16A may communicate via wireless communication channel 26A in the manner described above. Processor 36 may interact with telemetry circuit 42 to receive data from programming device 12A. In some instances, processor 36 may receive via telemetry circuit 42 data indicating the above described selected channel on which to establish the wireless communication session. Processor 36 may, based on this received selected channel, configure telemetry circuit 42 to listen for data from remote programmer device 12A on this selected channel, thereby establishing a wireless telemetric communication session over the selected one, e.g., wireless communication channel 26A, of the plurality of wireless communication channels, e.g., the 10 channels described above. Processor 36 may then, upon receiving this data, wireless communicate data stored to memory 38 via wireless communication channel 26A to remote programmer device 12A. As a result, the wireless communication session may be referred to as “wireless telemetry,” as the session relates to monitoring of data. In this manner, IMD 16A enables a programming device, such as programming device 12A, to wirelessly communication with IMD 16A.

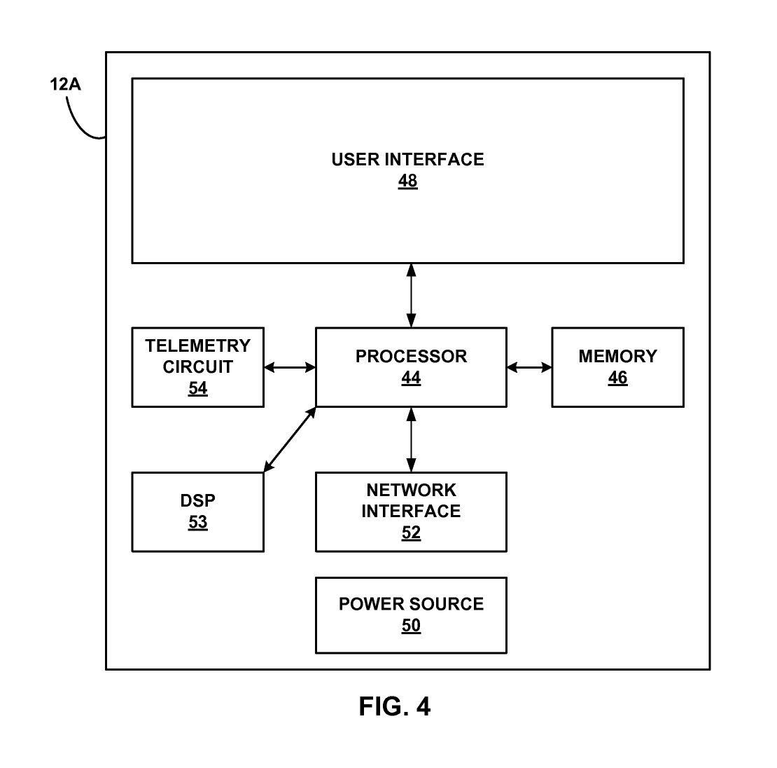

FIG. 4 is a functional block diagram illustrating various components of a programming device 12A according to one example configuration. As shown in FIG. 4, programming device 12A includes a processor 44, a memory 46, a user interface 48, a power source 50, a network interface 52, a digital signal processor 53 (“DSP 53”), and a telemetry circuit 54. A clinician or patient 30 interacts with user interface 48 in order to manually define or change the stimulation parameters of a program, turn stimulation ON or OFF, view therapy information, view patient physiological parameter information, or otherwise communicate with a respective one or more of IMDs 16.

User interface 48 may include a screen and one or more input buttons that allow remote programmer device 12A to receive input from a user. In some examples, user interface 48 may additionally or alternatively utilize a touch screen display. The screen may be a liquid crystal display (LCD), dot matrix display, organic light-emitting diode (OLED) display, touch screen, or any other device capable of delivering and/or accepting information. Input buttons may include a touch pad, increase and decrease buttons, emergency shut off button, and other buttons needed to control the stimulation therapy, as described above. In some examples, a programming device 12 need not include user interface 48, such as home or patient monitors that facilitate remote interaction with an IMD 16 by a computing device 20, data storage device 22, or server via a network 18.

Processor 44 controls user interface 48, and retrieves data from and stores data to memory 46. Processor 44 also controls the transmission of data through telemetry circuit 54 to or from respective IMDs 16 and the transmission of data through network interface 52 to or from network 18. Memory 46 includes operation instructions for processor 44, data related to patient 30 therapy and physiological parameters, and data corresponding to ambient noise 14, as will be described in greater detail below. The operational instructions in memory cause processor 44 and programming device 12 to provide the functionality ascribed to them herein. Processor 44 may comprise any one or more microprocessors, digital signal processors (DSPs), application specific integrated circuits (ASICs), field programmable gate arrays (FPGAs), and/or discrete analog or digital logic circuitry. Memory 46 may comprise any one or more of random access memory (RAM), read-only memory (ROM), electronically-erasable programmable ROM (EEPROM), hard disk, CD-ROM or the like.

Power source 50 may be a rechargeable battery, such as a lithium ion or nickel metal hydride battery. Other rechargeable or conventional batteries may also be used. In some cases, programming device 12A may be used when coupled to an alternating current (AC) outlet, i.e., AC line power, either directly or via an AC/DC adapter.

Network interface 52 may comprise a module for interfacing with a network, such as network 18. Network interface 52 may implement the above described protocols to carry out communications over links 24 and network 18, such as IP and TCP. Although described above with respect to these packet-based protocols, network interface 52 may implement any communication protocol for communicating data via a network, such as network 18. In this respect, network interface 52 may interface with network 18 to enable programming device 12A to communicate data over network 18 to any other device coupled to network 18 via links 24.

DSP 53 represents a module capable of performing signal processing. DSP 53 may, as shown in FIG. 4, comprise a separate processor from processor 44. Alternatively, the signal processing attributed in this disclosure to DSP 53 may be implemented by processor 44. In other instances, DSP 53 may be included within telemetry circuit 54 or otherwise integrate with telemetry circuit 54. DSP 53 may process signals received by telemetry circuit 54 to pre-process those signals as described below in more detail.

Telemetry circuit 54 allows the transfer of data to and from one or more IMDs 16. Processor 44 may automatically control telemetry circuit 54 to communicate with one of IMDs 16, e.g., IMD 16A, at a scheduled time, or when telemetry circuit 54 detects the proximity of IMD 16A. In some examples, processor 44 may control telemetry circuit 54 to communicate with IMD 16A when signaled by a user through user interface 48. To support RF communication, telemetry circuit 54 may include appropriate electronic components, such as amplifiers, filters, mixers, encoders, decoders, and the like. Telemetry circuit 54 may also include one or more antennas by which to wirelessly communicate with respective IMDs 16. In some examples, telemetry circuit 54 includes a first and a second antenna. Telemetry circuit 54 may communicate this data via the above described MICS frequency range (e.g., the frequency range of 402 to 405 MHz). Telemetry circuit 54 may, via this frequency, communicate with another telemetry circuit, e.g., telemetry circuit 38, of IMD 16A, as shown in FIG. 3.

As described above, programming device 12A may monitor ambient noise 14A and transmit data corresponding to the monitored levels of ambient noise 14A to data storage device 22. To monitor ambient noise 14A, one of users 28 may, either directly or remotely via network interface 52, interact with user interface 48 to cause programming device 12A to scan one or more wireless communication channels. User 28A, for example, may specify a schedule, which is stored to memory 46, by which processor 44 activates telemetry circuit 54 and causes telemetry circuit 54 to scan the one or more wireless communication channels. As another example, user 28A may initiate the scan of the one or more wireless communication channels. User 28A may initiate the scan as a survey of devices 12 in a particular geographic region. In some instances, processor 44 may be configured to store the above described scan of the one or more wireless communication channels that occurs prior to connecting to one or more of IMDs 16 in memory 46. This pre-connection scan may be implemented as a listen before talk/least interfered channel, clear channel assessment algorithm.

Regardless of how the scan is initiated, telemetry circuit 54 may scan the one or more wireless communication channels. In some cases, processor 44 may control telemetry circuit 54 to scan a signal strength across each of the above described 220 kHz channels (or 300 kHz channels including the two guard bands). In some cases, processor 44 may control telemetry circuit 54 to scan the signal strength across each of the channels for each of the one or more (e.g., two) antennas of telemetry circuit 54. In other words, for each of the one or more antennas, processor 44 may control telemetry circuit 54 to scan the signal strength of each channel one or more times.

Processor 44 may control telemetry circuit 54 to take a configurable number of samples for each antenna/channel combination. For example, processor 44 may control telemetry circuit 54 to take a configurable 200 samples of a configurable 50 microseconds (μs) duration over a configurable 10 millisecond (ms) period of time for each antenna/channel combination. Each of these samples may represent a received signal strength indicator (RSSI) sample, which is a measure of the power present in a received radio signal on each wireless or MICS channel.

One of users 28 may configure these various quantities, durations and periods by, again, interacting with processor 44 either directly or remotely via network interface 52 or with user interface 48. Telemetry circuit 54 may forward the data corresponding to ambient noise, e.g., the RSSI samples, to processor 44, which may store this monitored data corresponding to ambient noise 14A to memory 46. Processor 44 may timestamp or otherwise mark each sample, e.g., within memory 46, to indicate a date and time that each sample was taken or determined. Processor 44 may also, either alternatively or in conjunction with storing the monitored data, forward this data to DSP 53.

DSP 53, upon receiving this monitored data, may perform signal processing on the monitored data to further refine the monitored data. That is, DSP 53 may average the data over time and generate metrics describing amplitude, phase shift, period and other signal characteristics. DSP 53 may, for example, determine, for each channel (or each antenna of telemetry circuit 54 or antenna/channel pair), an average of the samples, a standard deviation of the samples, min/max amplitude value for the samples, etc. DSP 53 may in this manner compress or otherwise reduce the amount of data that need be transmitted to a memory or other data storage medium external from programming device 12A. DSP 53 may, upon processing this information, forward the processed signal information back to processor 44, which in turn may store the information to memory 46 and/or forward the information to network interface 52.

In some cases, the data, either in raw form as monitored by telemetry circuit 54 or in processed form as processed or pre-processed by DSP 53, may be stored to memory 46. In other cases, where memory 46 does not comprise enough memory to store substantial amounts of data, only processed data may be stored to memory 46, while the raw data is forwarded to network interface 52 for transmission to data storage device 22 via network 18. In other instances, none of the data, either raw or processed, may be stored to memory 46, and instead processor 44 may forward either or both the raw and processed data to network interface 52 for transmission via network 18 to data storage network 18. In some cases, processor 44 may store data to memory 46 until transmission via network 18, after which the data may be overwritten or removed from memory 46.

Processor 44 may forward the data, either raw or processed, to network interface 52 for transmission to data storage device 22 via network 18 in a number of ways. In one instance, processor 44 may periodically retrieve data stored to memory 46 and forward the data to network interface 52 for transmission to data storage device 22. In another instance, processor 44 may, upon receiving either or both the raw or processed data, forward the data to network interface 54. In yet another instance, processor 44 may only forward the processed data to network interface 54 to conserve bandwidth. In this manner, programming device 12A may monitor ambient noise 14A occurring on one or more communication channels used for the wireless communication with an IMD, e.g., IMD 16A, and transmit the data, either raw or processed, corresponding to the monitored ambient noise to a data storage device external from the device, e.g., data storage device 22.

Processor 44 may also analyze telemetric sessions established by telemetry circuit 54 and store telemetric session data to memory 46 concerning these sessions. The telemetric session data may be included within the raw data that is sent via network interface 52 to a data storage device. In this respect, ambient noise data may also include telemetric session data. Telemetric session data may comprise the above described the above connections-dropped-to-connections-attempted metric.

Processor 44 may, with respect to this exemplary metric, maintain a number of connections dropped by telemetric circuit 54 in memory 46 and a number of connections attempted by telemetric circuit 54 in memory 46. Processor 44 may update these numbers after each dropped and attempted telemetric session, respectively, with an IMD 16. Processor 44 may calculate the metric by dividing the stored number of connections dropped by the stored number of connections attempted and include this information with the raw or processed data transmitted via network interface 52. Processor 44 may, in some cases, maintain a separate number of connections dropped and attempted for each IMD 16 for which a connection is attempted to further refine the metric, or alternatively, maintain a single number of connections dropped and attempted for all of respective IMDs 16.

Processor 44 may in some embodiments analyze the data, raw or processed, and issue alerts or warnings via user interface 48 to patient 30. For example, processor 44 may determine from a series of zero averages on each of the scanned channels that one or more of the antennas has malfunctions. Processor 44 may then present a warning or alert via user interface 48 to patient 30. Processor 44 may also forward the alert to a clinician programmer device, e.g., computing device 20, of the clinician responsible for the care of patient 30 via network interface 52, or to data storage device 22, which may forward the alert to computing device 20. Processor 44 may also perform more in depth analysis to determine that a source of ambient noise 14A causing high levels of interference may reside to close to remote programmer device 12A.

In this manner, programming devices 12A and, more particularly, processor 44 may implement the operations described above with respect to data analysis module 34. In this respect, processor 44 may implement those operations attributed to data analysis module 34 to locally diagnose or troubleshoot wireless communications in light of ambient noise 14A. Processor 44 may then issue an error, warning or other alert via user interface 48 to patient 30 and/or via network interface 52 to a clinician programming device, e.g., computing device 20, or data storage device 22. The techniques therefore should not be limited with respect to analysis and automatic troubleshooting to a device external to programming device 12A but may be implemented by each of programming devices 12.

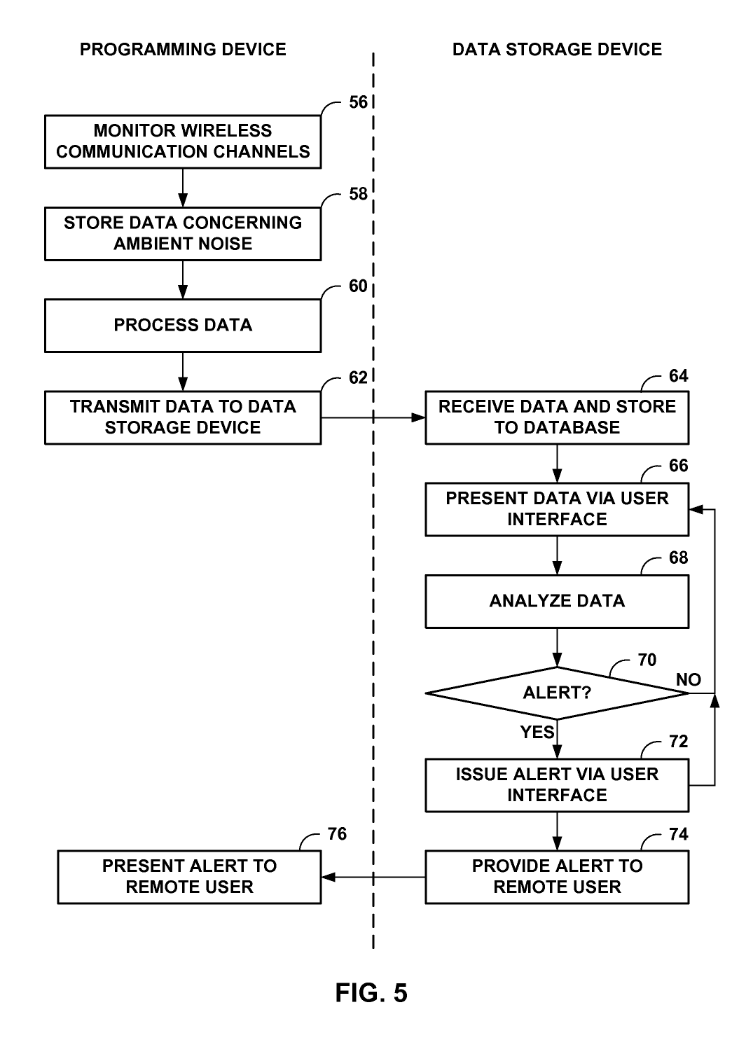

FIG. 5 is a flowchart illustrating an example interaction between a programming device, such as programming device 12A of FIG. 1, and a data storage device, such as data storage device 22. Initially, programming device 12A may monitor, in the above described manner, one or more wireless communication channels used to communicate with one or more of respective IMDs 16 (56). Programming device 12A may monitor the channels using telemetry circuit 54 of programming device 12A (FIG. 4). Programming device 12A may store this raw data concerning ambient noise 14A to an internal memory, such as memory 46 (58).

Programming device 12A may also pre-process this raw data using DSP 53 to generate processed data, also as described above (60). Programming device 12A may again store this processed data to memory. Programming device 12A may then transmit either or both the raw and processed data to a memory remote from programming device 12A, such as data storage device 22 (62). In particular, programming device 12A may, for example, utilize network interface 52 to transmit this data, in raw and/or processed format, via network 18 to data storage device 22.

Data storage device 22 may receive and store this data in a database as ambient noise data 30 (64). User interface module 32 of data storage device 22 may further present this data via one or more user interfaces with witch a user, such as user 28A, may interact to view or otherwise interact with ambient noise data 30 (66). Users 28 may view ambient noise data 30 to detect emerging noise patterns, as described above.

In some instances, data analysis module 34 of data storage device 22 may automatically analyze ambient noise data 30 to determine whether to alert user 28A of a malfunction or other issue with one of remote programmer devices 12 (68). These other issues may comprise a new or emerging noise pattern that interferes with one or more of the channels used for telemetric communication, e.g., the above described MICS channels.

If the analysis determines no error or malfunction has occurred (“NO” 70), user interface module 32 may continue to present ambient noise data 30 via a user interface (66). However, if an error or other issue is determined from the analysis (“YES” 70), data analysis module 34 may, as described above, cause user interface module 32 to present an alert, error, or other message via a user interface to user 28A (72). In this manner, ambient noise data 30 may facilitate troubleshooting of remote programmer devices 12, as well as enable users 28 to view and determine new and emerging noise patterns.

In some instances, user interface module 32 may also provide the alert, warning, or error to a remote user, such as user 28B or a clinician responsible for treating that patient in which the malfunctioning or otherwise interfered programming device 12 has been assigned (74). In other instances, user interface module 32 may provide the alert, warning or error to a patient via the malfunctioning or otherwise interfered programming device 12 via, for example, user interface 48. User interface 48 may present the alert, warning or error by way of a dedicated error light, which flashes in response to receiving the alert or by presenting a message or other message concerning the alert, warning or error via one of the above described display types (76). In this manner, a remote user, such as a clinician, a patient or other user 28B, may be alerted of the malfunction or interference and take appropriate measures to counteract or correct the alert, warning or error.

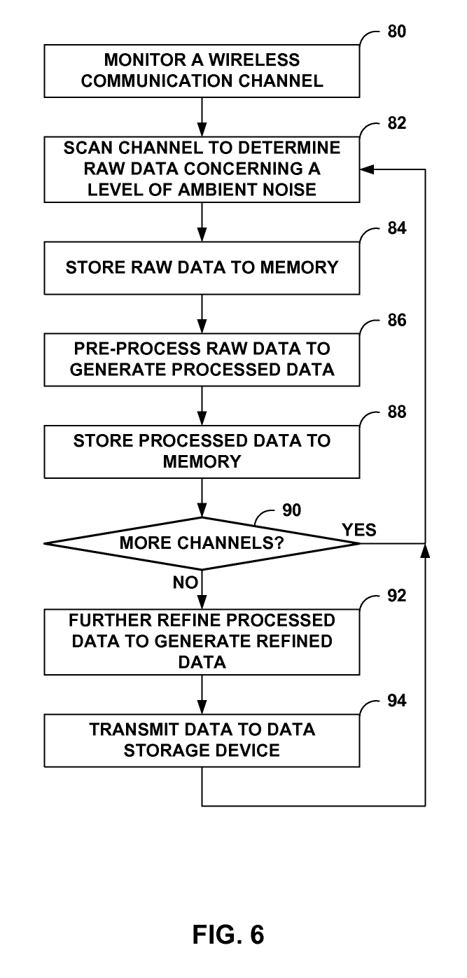

FIG. 6 is a flowchart illustrating an example operation of a programming device, such as programming device 12A of FIG. 4, in monitoring ambient noise. Initially, processor 44 may initiate the monitoring of wireless communication channels, such as channel 26A of FIG. 1, by causing telemetry circuit 54 to begin scanning at least one of a plurality of communication channels used for wireless communication (80). As described above, processor 44 may access a schedule stored to memory 46 to determine when to initiate this scan or may otherwise be configured to initiate the scan prior to establishing a wireless communication session with an IMD, such as IMD 16A. Processor 44 may, for example, initiate this scan (or a so-called “channel assessment”) every five seconds.

Telemetry circuit 54 may, in response to processor 44, scan at least one wireless communication channel to determine raw data corresponding or concerning the level of ambient noise 14A for that frequency sub-range used by that channel (82). Telemetry circuit 54, as described above, may forward this raw data to processor 44, which may store this raw data to memory 46 (84). The raw data may comprise, for example, 100 RSSI samples per antenna, or 200 RSSI samples in the case of two antennas.

Processor 44 may also forward this raw data to DSP 53 for processing. DSP 53 may process this raw data to generate processed data (86). For example, DSP 53 may determine a peak RSSI sample and use this peak RSSI sample to find the least interfered channel. DSP 53 may also calculate an average of the RSSI samples per antenna. Assuming the above exemplary configuration where 100 RSSI samples are taken per channel, DSP 52 may calculate an average of 100 RSSI samples per antenna. In some instances, DSP 53 may determine these two averages per channel (in the case of two antennas) without disrupting its scan of the least interfered channel. DSP 53 may return these averages to processor 44 which may store these averages to memory 46 (88).

During or after the above processing, processor 44 may determine whether to scan additional wireless communication channels (90). As described above, a total of 10 channels may be used for telemetric wireless communication according to the above mentioned MICS. Thus, telemetry circuit 54 may scan each of these 10 channels to determine raw data corresponding to a level of ambient noise 14A across each of these channels, DSP 53 may process this raw data to generate processed data, and processor 44 may store this raw and processed data corresponding to each channel to memory 46 (80-88). That is, processor 44 may, for example, receive 200 RSSI samples per channel for a total of 2000 RSSI samples and store these 2000 RSSI samples to memory 46. Again, assuming two antennas, DSP 20 may determine two averages per channel for a total of 20 averages.

After scanning each channel, processor 44 may further refine these averages by retrieving the 20 averages and past averages from memory 46 and forwarding these 20 averages and past averages to DSP 53 for further processing. DSP 53 may further refine this processed data by averaging these averages to generate refined data (92). DSP 53 may forward this refined data to processor 44. Processor 44 may, at some point, forward the raw, processed and/or refined data to network interface 52 for transmission via network 18 to data storage device 22 (94).

Various examples have been described. These and other examples are within the scope of the following claims.

1. A method comprising:
monitoring, with a device that wirelessly communicates with at least one implantable medical device (IMD), an ambient noise on one or more communication channels used for the wireless communication with the at least one IMD; and
transmitting data corresponding to the monitored ambient noise via a network to a data storage device remote from the device that wirelessly communicates with the at least one IMD.

2. The method of claim 1, wherein monitoring the ambient noise comprises monitoring the ambient noise on a plurality of communication channels included within a frequency range of 402 to 405 megahertz (MHz).

3. The method of claim 2, wherein monitoring the ambient noise further comprising monitoring the ambient noise on ten communication channels, each channel including two 40 kilohertz (kHz) guard bands and a 220 kHz sub-range of the frequency range.

4. The method of claim 1,
wherein monitoring the ambient noise comprises sampling the one or more communication channels to determine a receive signal strength indicator (RSSI) sample that measures a power present on the one or more communication channels for a discreet instance in time, and
wherein the data comprises the RSSI sample.

5. The method of claim 1, wherein the data comprises raw data corresponding to the monitored ambient noise on the one or more communication channels and processed data generated from the raw data, the method further comprising:
storing the raw data to a memory internal to the device;
processing the raw data to generate processed data; and
storing the processed data to the memory internal to the device.

6. The method of claim 5,
wherein the raw data comprises a plurality of samples of a signal received over each of the one or more communication channels,
wherein processing the raw data comprises determining at least one average of the plurality of samples for each of the one or more communication channels, and
wherein storing the processed data comprises storing the at least one average of the plurality of samples for each of the one or more communication channels to the memory internal to the device.

7. The method of claim 1,
wherein the device comprises one of a home monitor device and a home programming device,
wherein the data storage device comprises a network server that stores the data to a database hosted by the network sever.

8. The method of claim 1, further comprising:
storing with the data storage device, the data corresponding to the monitored ambient noise;
analyzing, with the data storage device, the stored data corresponding to the monitored ambient noise; and
presenting with the data storage device, an alert based on the analysis via a user interface.

9. The method of claim 1, further comprising:
storing, with the data storage device, the data corresponding to the monitored ambient noise;
analyzing, with the data storage device, the stored data corresponding to the monitored ambient noise; and
providing, with the data storage device, an alert based on the analysis to a remote user via the device that wirelessly communicates with the IMD.

10. The method of claim 9, wherein the device that wirelessly communicates with the IMD includes a user interface to present the alert to a patient.

11. A medical device for wirelessly communicating with at least one implantable medical device (IMD), the medical device comprising:
a telemetry circuit that monitors an ambient noise on one or more communication channels used for the wireless communication with the at least one IMD; and
a network interface that transmits data corresponding to the monitored ambient noise via a network to a data storage device remote from the device that wirelessly communicates with the at least one IMD.

12. The medical device of claim 11, wherein the telemetry circuit monitors the ambient noise by monitoring the ambient noise on a plurality of communication channels included within a frequency range of 402 to 405 megahertz (MHz).

13. The medical device of claim 12, wherein the telemetry circuit monitors the ambient noise by monitoring the ambient noise on ten communication channels, each channel including two 40 kilohertz (kHz) guard bands and a 220 kHz sub-range of the frequency range.

14. The medical device of claim 11,
wherein the telemetry circuit monitors the ambient noise by sampling the one or more communication channels to determine a receive signal strength indicator (RSSI) sample that measures a power present on the one or more communication channels for a discreet instance in time, and
wherein the data comprises the RSSI sample.

15. The medical device of claim 11, wherein the data comprises raw data corresponding to the monitored ambient noise on the one or more communication channels and processed data generated from the raw data, the medical device further comprising:
a processor that processes the raw data to generate processed data; and
a memory that stores the raw data and the processed data.

16. The medical device of claim 15,
wherein the raw data comprises a plurality of samples of a signal received over each of the one or more communication channels,
wherein the processor processes the raw data by determining at least one average of the plurality of samples for each of the one or more communication channels, and
wherein the memory stores the processed data by storing the at least one average of the plurality of samples for each of the one or more communication channels.

17. The medical device of claim 11,
wherein the medical device comprises one of a home monitor device and a home programming device,
wherein the data storage device comprises a network server that stores the data to a database hosted by the network sever.

18. The medical device of claim 11, further comprising a user interface module,
wherein the network interface receives an alert based on an analysis of the data performed by the data storage device, and
wherein the user interface module presents the alert via a user interface.

19. A system comprising:
at least one implantable medical device (IMD);
a network;
a data storage device; and
a medical device for wirelessly communicating with the at least one IMD, the medical device comprising:
a telemetry circuit that monitors an ambient noise on one or more communication channels used for the wireless communication with the at least one IMD; and
a network interface that transmits data corresponding to the monitored ambient noise via the network to the data storage device external from the device that wirelessly communicates with the at least one IMD.

20. The system of claim 19, wherein the telemetry circuit monitors the ambient noise by monitoring the ambient noise on a plurality of communication channels included within a frequency range of 402 to 405 megahertz (MHz).

21. The system of claim 20, wherein the telemetry circuit monitors the ambient noise by monitoring the ambient noise on ten communication channels, each channel including two 40 kilohertz (kHz) guard bands and a 220 kHz sub-range of the frequency range.

22. The system of claim 19,
wherein the telemetry circuit monitors the ambient noise by sampling the one or more communication channels to determine a receive signal strength indicator (RSSI) sample that measures a power present on the one or more communication channels for a discreet instance in time, and
wherein the data comprises the RSSI sample.

23. The system of claim 19, wherein the data comprises raw data corresponding to the monitored ambient noise on the one or more communication channels and processed data generated from the raw data, the device further comprising:
a processor that processes the raw data to generate processed data; and
a memory that stores the raw data and the processed data.

24. The system of claim 23,
wherein the raw data comprises a plurality of samples of a signal received over each of the one or more communication channels,
wherein the processor processes the raw data by determining at least one average of the plurality of samples for each of the one or more communication channels, and
wherein the memory stores the processed data by storing the at least one average of the plurality of samples for each of the one or more communication channels.

25. The system of claim 19,
wherein the medical device comprises one of a home monitor device and a home programming device,
wherein the data storage device comprises a network server that stores the data to a database hosted by the network sever.

26. The system of claim 19, further comprising:
a memory that stores with the data storage device, the data corresponding to the monitored ambient noise;
analyzing, with the data storage device, the stored data corresponding to the monitored ambient noise; and
presenting with the data storage device, an alert based on the analysis via a user interface.

27. The system of claim 19, wherein the data storage device comprises:
a memory that stores the data corresponding to the monitored ambient noise;
a data analysis module that analyzes the stored data corresponding to the monitored ambient noise; and
a user interface module that presents an alert based on the analysis via a user interface.

28. The system of claim 19, wherein the data storage device comprises:
a memory that stores the data corresponding to the monitored ambient noise;
a data analysis module that analyzes the stored data corresponding to the monitored ambient noise; and
a user interface module that provides an alert based on the analysis to a remote user via the device that wirelessly communicates with the IMD.

29. The system of claim 28, wherein the device that wirelessly communicates with the IMD includes a user interface module to present the alert to a patient via a user interface.

30. A computer-readable medium comprising instructions that cause a programmable processor to:
monitor, with a device that wirelessly communicates with at least one implantable medical device (IMD), an ambient noise on one or more communication channels used for the wireless communication with the at least one IMD; and
transmit data corresponding to the monitored ambient noise via a network to a data storage device remote from the device that wirelessly communicates with the at least one IMD.

31. The computer-readable medium of claim 30, wherein the data comprises raw data corresponding to the monitored ambient noise on the one or more communication channels and processed data generated from the raw data, the instruction further causing the processor to:
store the raw data to a memory internal to the device;
process the raw data to generate processed data; and
store the processed data to the memory internal to the device.

32. The computer-readable medium of claim 30, wherein the instructions further cause the processor to:
receive an alert based on an analysis of the data performed by the data storage device; and
present the alert via a user interface.
